(19) United States
(12) Patent Application Publication
(10) Pub. No.: US 2011/0020846 A1
(43) Pub. Date: Jan. 27, 2011

(54) Oral cancer biomarker and inspection method using the same

(76) Inventors: Jau-Song Yu, Kwei-Shan (TW); Li-Ping Weng, Kwei-Shan (TW); Ling-Ling Hsieh, Kwei-Shan (TW); Chih-Ching Wu, Kwei-Shan (TW); Ying Liang, Kwei-Shan (TW); Ching-Ping Tseng, Kwei-Shan (TW); Bao-Lian Hsu, Kwei-Shan (TW); Lang-Ming Chi, Kwei-Shan (TW)

Correspondence Address: Muncy, Geissler, Olds & Lowe, PLLC, 4000 Legato Road, Suite 310, FAIRFAX, VA, 22033, US

(21) Appl. No.: 12/460,656

(22) Filed: Jul. 22, 2009

(51) Int. Cl. G01N 33/53; C07K 14/00

(52) U.S. Cl. 435/7.92; 530/395

(57) ABSTRACT

The present invention discloses an oral cancer biomarker and an inspection method using the same. The biomarker is Mca-2 binding protein (Mac-2BP), which can be directly detected in the specimen of the body fluid of a testee, and which can realize a fast and effective clinical diagnosis of oral cancer.

### FIELD OF THE INVENTION

The present invention relates to a cancer biomarker and an inspection method, particularly to an oral cancer biomarker and an inspection method using the same.

### BACKGROUND OF THE INVENTION

Oral cancer is the 11th most common human neoplasm in the world and is a complex disease arising in various organs, including tongue, buccal, hypopharynx, oropharynx, gum, palate, lips, and the floor of the mouth. Different parts of the tumor have distinct clinical presentations and outcomes, and are treated with different strategies. More than 90% of oral cancer cases are oral squamous cell carcinomas (OSCC), which are associated with a very poor prognosis. Previous studies have indicated the involvement of multiple genetic, epigenetic and metabolic changes in the evolution of OSCC, and these changes are strongly associated with environmental carcinogens such as tobacco, alcohol and betel quid chewing. In Taiwan, approximate 85% of OSCC patients have the custom of betel quid chewing, which has been suspected to be involved in the etiology of OSCC. Approximately 50-70% of OSCC patients die within 5 years of diagnosis, mainly due to local recurrence, metastasis to the esophagus or lungs, and/or the development of additional primary cancers. Late presentation, lack of suitable markers for early detection and failure of advanced lesions to respond to chemotherapy contribute to the poor outcome of this cancer. The overall 5-year survival rate and morbidity for patients with OSCC has not improved over the past two decades, and the World Health Organization predicts that the incidence of oral cancer will continuously increase worldwide, extending this trend into the next several decades.

Currently, OSCC is diagnosed through physical examination and excisional biopsies, and the treatment strategies rely on traditional surgery, radiotherapy, and chemotherapy. Radiologic or physical examination requires 1 to 2 cm of tumor mass for detection, and the clinical stages of OSCC determine the severity and prognosis of the cancer. Unfortunately, one study indicated that more than 50% of oral cancer patients in Taiwan presented with stage III or stage IV tumors. Despite the notable advantage of earlier diagnosis of head and neck cancers, and the fact that visual inspection or dye staining of the mouth can be useful for early detection of oral cancer and precancerous lesions, there is no currently accepted strategy for early diagnosis of OSCC.

Regarding biomarker research for OSCC, although studies have identified altered expression levels of many gene products in OSCC tissues, such gene products have yielded negligible definitive prognostic or predictive information to date. Recently, genomic (microarray) techniques have been used to identify the genes and molecular pathways involved in the progression of oral cancer, in an effort to support better classification of normal, pre-malignant and OSCC specimens, or improved prediction of patient outcomes. In contrast, relatively few studies have sought to systematically identify protein biomarkers for OSCC. Some studies have used 2D-gel protein profiling to identify proteins showing differential expression in OSCC tissue specimens. Subsequent protein identification using mass spectrometric analysis has led to the identification of approximately 40 proteins that are differentially expressed in OSCC tissues, but these proteins are not necessarily detectable in-accessible body fluids, such as plasma, serum or urine. For practical usage in tumor screening, biomarkers should be measurable in body fluid samples.

Thus, the present invention proposes an oral cancer biomarker and method for detecting oral cancer to overcome the abovementioned problems.

### SUMMARY OF THE INVENTION

The primary objective of the present invention is to provide an oral cancer biomarker and an inspection method using the same, wherein the oral cancer biomarker can be directly detected in the specimen of the body fluid of a testee and thus can realize a fast and effective clinical diagnosis of oral cancer.

To achieve the abovementioned objective, the present invention proposes a biomarker for oral cancer diagnosis—Mca-2 binding protein (Mac-2BP), which is proved to exist in the body fluid of testees.

The present invention also proposes an oral cancer inspection method, which detects the Mac-2BP expression level of the body fluids of the testees suspected to have oral cancer.

The present invention further proposes an oral cancer inspection method using an oral cancer biomarker, which comprises steps: cultivating cell lines of oral cancer in a serum-free environment, and respectively collecting the proteins secreted by the cell lines; using 9-15% gradient electrophoresis to separate the proteins and staining the proteins with silver ion; cutting off the electrophoresis gel containing stained proteins, and using trypsin to hydrolyze in-gel proteins; using MALDI-TOF (Matrix-Assisted Laser Desorption/Ionization-Time Of Flight) to analyze the hydrolyzed proteins to identify the identities of the proteins respectively secreted by the cell lines; performing analysis to find out the proteins that have been identified in different cell lines simultaneously and using the proteins identified in different cell lines simultaneously as biomarkers.

Below, the embodiments are described in detail to make easily understood the objective, characteristics and accomplishes of the present invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 Flow chart of the strategy used to identify potential OSCC markers on the basis of cancer cell secretome analysis.

FIG. 2. SDS-PAGE analysis of conditioned media from two OSCC cell lines. (A) The conditioned media of SCC4 and OEC-M1 cells (25 μg protein) were resolved on 9-15% gradient SDS gels and silver stained. (B) The viability of SCC4 and OEC-M1 cells grown for 24 h in complete (Com) or serum-free (SF) media was assayed as described in Materials and Methods. (C) Western blot analysis (20 μg protein) of the conditioned media (CM) and cell extracts (CE) from both cell lines, using an anti-β-tubulin antibody.

FIG. 3. Confirmation of secreted proteins by Western blot analysis.

FIG. 4. Overexpression of Mac-2 BP in OSCC tissues.

FIG. 5. Elevated Mac-2 BP levels in OSCC serum samples.

FIG. 6. Receiver operating characteristic (ROC) curve analysis of the diagnostic efficacy of Mac-2 BP in discriminating oral cancer patients from healthy controls.

### DETAILED DESCRIPTION OF PREFERRED EMBODIMENTS

The present invention adopts Mac-2 BP binding protein (Mac-2BP) as a biomarker for detecting oral cancer.

### Mac-2 Binding Protein (Mac-2 BP)

Mac-2 BP is a secreted glycoprotein of 90-100 kDa, originally discovered as a tumor-associated antigen 90K (1, 2) and as a ligand of galectin-3 (formerly Mac-2) (3, 4). The functions of Mac-2 BP are not yet fully understood, although it is known to enhance cell-cell and cell-extracellular matrix adhesion (5) and induce production of IL-1, IL-6, and other cytokines from blood monocytes (4). Elevated expression levels of Mac-2 BP have been observed in tissues and sera of patients with different types of cancer, including breast cancer (2), non-Hodgkin's lymphoma (6), ovarian cancer (7), lung cancer (8), colon cancer (9) and NPC (10). Several evidences support that endogenous ligands of galectins including laminin, fibronectin, lysosome-associated membrane proteins and Mac-2 BP have been reported the altered expression in various cancer type were associated with patients clinical outcome. Mac-2BP was found as a tumor-associated antigen in human breast cancer originally and mostly expressed on the surface of tumor cells. It is synthesized and secreted in many cell type and serum level of Mac-2 BP in patient's peripheral blood have been found elevated in several human disease including infection by hepatitis B virus, hepatitis C virus (11), human immunodeficiency virus (12) and cancers. The level of high Mac-2 BP is associated with a poor prognosis (13-15). In a previously study of 310 patients with breast cancer, Mac-2 BP serum level was not correlated with tumor size, tumor histology or estrogen receptor status, but strongly associated with liver metastasis (16). Similarly, its expression was significantly associated with worse outcome and distant metastasis in stage I non-small cell lung cancers (17).

Although the following detailed description contains many specific details for the purposes of illustration, anyone of ordinary skill in the art will appreciate that many variations and alterations to the following details are within the scope of the invention. Accordingly, the examples of embodiments of the invention described below are set forth without any loss of generality to, and without imposing limitations upon, the claimed invention.

### EXAMPLE

Identification of Proteins Released from the two OSCC Cell Lines

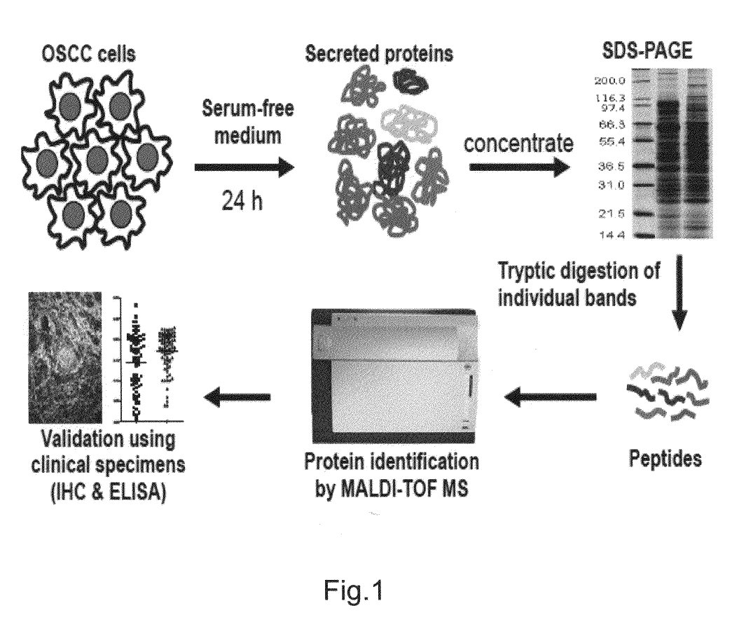

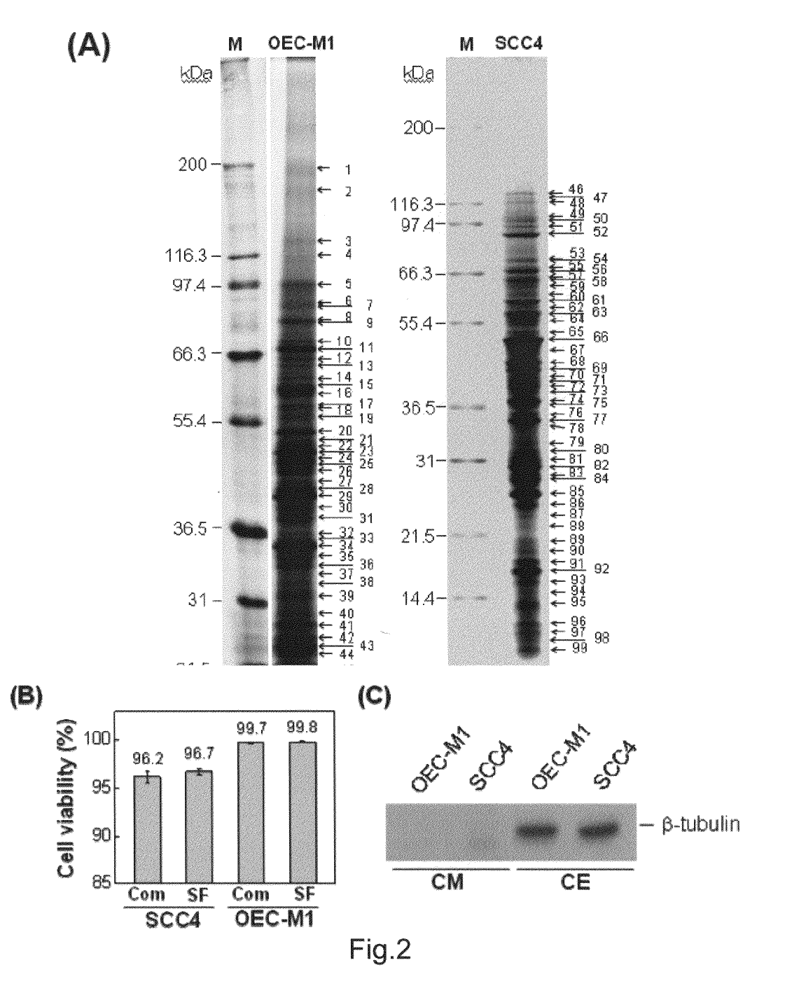

The inventor used a secretome-based strategy to identify potential OSCC biomarker(s) that might be detectable in body fluids such as serum or plasma. FIG. 1 denotes the schematic diagram of this strategy. In FIG. 2A, the inventor cultured two OSCC cell lines, OEC-M1 and SCC4, in serum-free medium for 24 hr, collected the conditioned media, and analyzed their protein profiles using 9-15% SDS-PAGE followed by silver staining. Protein bands were marked, numbered, and excised for further protein identification using MALDI-TOF mass spectrometry. Lane ‘M’ denotes molecular weight markers. In FIG. 2B, both cell lines grew continuously in serum-free medium, and the viability of both cell lines remained >96% following incubation in serum-free medium for 24 hr. The results showed that serum-starvation for 24 h had a little effect on the viability of the two OSCC cell lines. The relative distribution of β-tubulin, an abundant cytosolic protein, in the conditioned media and in the extracts of residual cells attached on the culture dishes was examined by Western blot analysis. As shown in FIG. 2C, β-tubulin was detected in the total cell extracts but not in the conditioned media, suggesting that the release of proteins into the conditioned media was not caused by cell lysis. The protein bands were individually excised, in-gel digested with trypsin, and analyzed by MALDI-TOF MS. The resulting peptide mass fingerprints were used to search protein identities against the NCBInr database, with the help of the Mascot engine. A total of 37 proteins were identified (Table 1); among them, 27 proteins were detected from OEC-M1 cells and 23 from SCC4 cells, and 17 proteins were detected in both cell lines. The SignalP 3.0 and SecretomeP 2.0 bioinformatics programs predicted that 19 of the identified proteins (51.4%) were likely to be secreted proteins (Table 1). In addition, published reports indicated that 11 of the proteins (29.5%) could be released from cells by the exosome pathway, a non-classical secretion mechanism (18-20) (Table 1). Overall, these analyses predicted that ˜80% of the MS-identified proteins could be secreted from OSCC cells, and also suggested that the strategy used here could be an appropriate approach for enriching and identifying the secretome of OSCC cell lines.

Among the 17 proteins identified in both OSCC cell lines, 14 had been previously reported as being dysregulated in certain cancer types (Table 2). It is interesting to note that six of the 14 proteins (moesin, alpha enolase, fascin, glutathione s-transferase P, peroxiredoxin 1 and 14-3-3 zeta) were previously demonstrated as being overexpressed in oral cancer tissues in studies using immunohistochemistry, ELISA and/or Western blot analysis (Table 2). In addition, six proteins (heat shock protein 90, pyruvate kinase isozymes M1/M2, alpha enolase, glyceraldehyde 3-phophate dehydrogenase, triosephosphate isomerase and glutathione s-transferase P) were recently shown to be up-regulated in OSCC tissues by mass spectrometry-based proteomic approaches (21, 22, 23, 24). These observations suggest that identification of the proteins selectively enriched in the secretome of OSCC cell lines could be an efficient and convenient strategy for discovering proteins overexpressed in OSCC.

TABLE 1
Oral Cancer Cell-Secreted Proteins Identified by MALDI-TOF
MS

| | | Band no.^{b} (score^{c}/% seq | | | | |
|---|---|---|---|---|---|---|
| | | cov^{d}/no. of masses | | | | SignalP |
| | Accession | matched) | | Protein | HMM | secretomeP |
| Protein identified | number^{a} | OEC-M1 | SCC4 | ontology^{e} | probaility^{f} | NN-score^{g} |
| GTPase-activating | Q96FS4 | 1 (82/17%/ | | Signaling | 0.004 | 0.375 |
| protein Spa-1 | | 12) | | | | |
| Thrombospondin-1^{h} | P07996 | 2 (94/13%/ | | Cell | 0.994 | 0.345 |
| | | 16), 37, 38 | | adhesion | | |
| Protein tyrosine | Q86WS0 | 3 (79/17%/ | | Signaling | 1.000 | 0.420 |
| phosphatase, receptor | | 21) | | | | |
| type F | | | | | | |
| Sulfhydryl oxidase 1 | O00391 | 6 (77/24%/ | | Enzyme | 1.000 | 0.611 |
| (Quiescin Q6) | | 14) | | | | |
| Mac-2-binding | Q08380 | 6 (71/24%/ | 46 (75/24%/ | Cell | 1.000 | 0.738 |
| proteinⁱ | | 13), 4 | 12), 47~51 | adhesion | | |
| Fibronectin 1^{h} | P02751 | 5 (107/15%/ | | Cell | 0.997 | 0.371 |
| | | 24) | | adhesion | | |
| Heat shock protein | P07900 | 7 (74/25%/ | 52 (124/28%/ | Protein | 0.000 | 0.173 |
| 90-alpha^{h} | | 17) | 21) | folding | | |
| BiP protein^{h} | P11021 | 10 (110/39%/ | 53 (187/48%/ | Protein | 1.000 | 0.745 |
| | | 22) | 24), 68 | folding | | |
| Moesin^{h} | P26038 | 11 (90/35%/ | 54 (134/39%/ | Protein | 0.000 | 0.530 |
| | | 19) | 24) | folding | | |
| Disulfide-isomerase | P30101 | | 55 (121/37%/ | Enzyme | 1.000 | 0.707 |
| ER60^{h} | | | 16) | | | |
| HSP70 family HSPA8 | Q961S6 | 13 (111/44%/ | 65 (114/27%/ | Protein | 0.000 | 0.129 |
| protein^{h} | | 18), 12 | 16) | folding | | |
| HSP70-2^{h} | P08107 | | 57 (103/40%/ | Protein | 0.049 | 0.280 |
| | | | 14), 58 | folding | | |
| TGF beta-induced | Q15582 | 15 (150/43%/ | 56 (67/24%/ | Cell | 1.000 | 0.454 |
| protein BIGH3^{h} | | 22), 16, | 12) | adhesion | | |
| | | 17, 20 | | | | |
| Pyruvate kinase | P14618 | 18 (64/26%/ | 61 (178/36%/ | Metabolism | 0.089 | 0.420 |
| isozymes M1/M2^{h} | | 10) | 22), 62 | | | |
| Ezrin^{h} | P15311 | | 63 (84/19%/ | Protein | 0.000 | 0.563 |
| | | | 13) | folding | | |
| Fascin | Q16658 | 20 (67/39%/ | 64 (152/38%/ | Protein | 0.001 | 0.385 |
| | | 12) | 16) | folding | | |
| Glutathion synthase | P48637 | 21 (82/35%/ | | Enzyme | 0.000 | 0.484 |
| | | 11) | | | | |
| Cathepsin D^{h} | P07339 | 22 (61/24%/ | | Enzyme | 1.000 | 0.758 |
| | | 8) | | | | |
| Alpha enolase^{h} | P06733 | 23 (146/49%/ | 76 (64/21%/ | Enzyme | 0.000 | 0.536 |
| | | 18), 22 | 9), 66, 73 | | | |
| Plasminogen activator | P05121 | 26 (127/60%/ | | Protein | 0.999 | 0.644 |
| inhibitor-1 | | 17), 24, | | folding | | |
| | | 25 | | | | |
| Phosphoglycerate | P00558 | 28 (109/36%/ | 70 (68/36%/ | Metabolism | 0.000 | 0.389 |
| kinase 1^{h} | | 11), 29 | 10) | | | |
| PKCq-ineracting | O76003 | 29 (75/40%/ | | Protein | 0.182 | 0.542 |
| protein PICOT | | 11) | | folding | | |
| Fructose-bisphosphate | P04075 | 31 (92/50%/ | 71 (69/28%/ | Metabolism | 0.000 | 0.356 |
| aldolase A^{h} | | 14), 30 | 7), 72 | | | |
| Glyceraldehyde | P04406 | 32 (68/36%/ | 74 (80/32%/ | Enzyme | 0.000 | 0.467 |
| 3-phophate | | 7) | 11), 75 | | | |
| dehydrogenase^{h} | | | | | | |
| Nebulin | Q14215 | 35 (84/36%/ | | others | 0.000 | 0.224 |
| | | 35) | | | | |
| Tropomyosin alpha-4 | P67936 | | 79 (73/38%/ | Protein | 0.000 | 0.417 |
| chain isoform 2 | | | 13) | folding | | |
| 14-3-3 protein sigma | P31947 | | 81 (84/48%/ | Signaling | 0.000 | 0.345 |
| | | | 11) | | | |
| Heat shock 27 kDa | Q96E17 | | 83 (70/36%/ | Protein | 0.000 | 0.731 |
| protein 1 | | | 8) | folding | | |
| 14-3-3 protein zata, | P63104 | 40 (59/44%/ | 82 (75/35%/ | Signaling | 0.000 | 0.252 |
| chain A^{h} | | 9) | 11) | | | |
| Triosephosphate | P60174 | 39 (80/44%/ | 84 (107/44%/ | Metabolism | 0.013 | 0.390 |
| isomerase^{h} | | 10), 41 | 12), 98 | | | |
| Glutathione | P09211 | 42 (96/53%/ | 85 (103/56%/ | Enzyme | 0.084 | 0.545 |
| s-transferase P^{h} | | 11) | 8) | | | |
| Peroxiredoxin-1 | Q06830 | | 87 (83/41%/ | Enzyme | 0.000 | 0.528 |
| | | | 9) | | | |
| Neutrophil | P80188 | 44 (75/51%/ | 86 (48/33%/ | others | 1.000 | 0.924 |
| gelatinase-associated | | 9), 43 | 5) | | | |
| lipocalin | | | | | | |
| Nucleoside | Q08WT6 | | 91 (58/38%/ | Enzyme | 0.000 | 0.514 |
| diphosphate kinase, | | | 5) | | | |
| chain R | | | | | | |
| Peptidylprolyly | P62937 | 45 (84/58%/ | 93 (43/30%/ | Protein | 0.001 | 0.339 |
| isomerase A | | 10) | 4), 92 | folding | | |
| (cyclophilin A)^{h} | | | | | | |
| Profilin chain A^{h} | P07737 | | 95 (54/39%/ | Protein | 0.000 | 0.469 |
| | | | 5) | folding | | |
| Tetraubiquitin | Q9ZSW0 | | 99 (89/81%/ | unknown | 0.001 | 0.477 |
| | | | 8) | | | |

^{a}Swiss-Prot accession numbers of identified proteins.
^{b}Numbering of the protein bands corresponds to that in FIG. 1.
^{c}Mascot scores of proteins identified by peptide mass fingerprints.
^{d}Percent sequence coverage (% seq cov) of matched peptides in the identified proteins.
^{e}The ontologies of identified proteins were analyzed using the Java application, GoMiner.
^{f}The signal peptides were predicted using the hidden Markov model of SignalP 3.0.
^{g}The nonclassical secretion of proteins was evaluated by the neural network output score of SecretomeP 2.0.
^{h}The protein has been reported to be present in exosomes.
ⁱProtein identified in both cell lines are denoted in bold.

TABLE 2
OSCC Cell-Secreted Proteins Known to Be Dysregulated in
Other Cancer Types

| Protein identified | Cancer type (detection method^{a})^{( Ref.No.)b} |
|---|---|
| Thrombospondin-1 | Cervical cancer (RT-PCR),³⁴ prostate cancer (IHC),^{35,37} |
| | colorectal cancer (IHC),³⁸ gastric cancer (IHC),³⁹ lung |
| | cancer (IHC),⁴⁰ breast cancer (IHC),⁴¹ bladder cancer |
| | (IHC),⁴² head and neck cancer (ELISA)⁴³ |
| Mac-2 binding protein | Pancreatic cancer (ELISA),^{44,45} lung cancer (IHC),⁴⁶ |
| | hepatocellular carcinoma (ELISA),⁴⁷ nasopharyngeal |
| | carcinoma (IHC, ELISA),⁴⁸ prostate cancer (IHC),⁴⁹ colon |
| | cancer (IHC),⁵⁰ gastric cancer (IHC)⁵¹ |
| Fibronectin | Gastric cancer (IHC),⁵² ovarian cancer (IHC),⁵³ breast |
| | cancer (IHC),^{54,55} gastrointestinal cancer(ELISA),⁵⁶ head |
| | and neck cancer (ELISA),⁵⁶ laryngeal cancer (IHC)⁵⁷ |
| Moesin | Ovarian adenocarinoma (cDNA microarray, IHC),⁵⁸, oral |
| | cancer (IHC)⁵⁹ |
| Heat shock protein 90 | Bladder cancer (IHC),⁶⁰ prostate cancer (IHC),⁶¹ breast |
| | cancer (IHC)⁶² |
| TGF β-induced protein BIGH3 | Colorectal carcinoma(Q-PCR),⁶³ pancreatic cancer(NB),⁶⁴ |
| | esophageal squamous carcinoma (cDNA microarray)⁶⁵ |
| Alpha enolase | Hepatocellular carcinoma(WB, IHC),⁶⁶ lung cancer(IHC),⁶⁷ |
| | oral cancer (IHC)⁶⁸ |
| Fascin | Ovarian cancer(IHC),⁶⁹ panceratic adenocarcinoma (cDNA |
| | microarray),⁷⁰ lung cancer(IHC),⁷¹ astrocytoma(IF, WB),⁷² |
| | breast cancer (IHC),⁷³ colorectal cancer (IHC),⁷⁴ renal |
| | cell carcinoma(IHC),⁷⁵ esophgeal carcinoma(IHC),⁷⁶ oral |
| | cancer (IHC)⁷⁷ |
| Plasminogen activator | Breast cancer(IHC, ELISA),^{78,79} lung cancer(IHC),⁸⁰ |
| inhibitor 1 | gastric cancer (IHC),^{81,86} colorectal cancer (IHC),⁸² |
| | head and neck cancer (NB),^{83,87} esophageal squamous |
| | cell carcinnoma (RT-PCR),⁸⁴ nasopharyngeal |
| | carcinoma (IHC, ELISA)⁸⁵ |
| Glutathione s-transferase P | Oral cancer (ELISA),⁸⁸ lung cancer (ELISA),^{89,90} gastric |
| | cancer(IHC),⁹¹ bladder cancer(ELISA),⁹² nasopharyngeal |
| | cancer (IHC),⁹³ breast cancer (IHC),⁹⁴ prostate cancer |
| | (IHC)⁹⁵ |
| Peroxiredoxin I | Oral cancer (IHC),⁹⁶ breast cancer (WB),⁹⁷ lung cancer |
| | (IHC, WB)^{98,99} |
| Phosphoglycerate kinase 1 | Lung cancer(IHC, ELISA, RT-PCR),^{100,101} pancreatic ductal |
| | adenocarinoma (IHC, ELISA),¹⁰² prostate cancer (ELISA)¹⁰³ |
| Fructose-bisphosphate aldolase A | Lung squamous carcinoma (IHC)¹⁰⁴ |
| 14-3-3 zeta | Lung cancer(RT-PCR, IHC),^{105,106} oral cancer(IHC, WB),¹⁰⁷ |
| | stomach cancer (2DE/MALDI-TOF MS)¹⁰⁸ |

^{a}Detection method: IHC, immunohistochemistry; Q-PCR, quantitative PCR; RT-PCR, reverse transcription-PCR; WB, Western blot; NB, Northern blot; 2DE, two-dimensional gel electrophpresis.
^{b}References are denoted in Supporting Information.

### Confirmation of Secreted Proteins by Western Blot Analysis

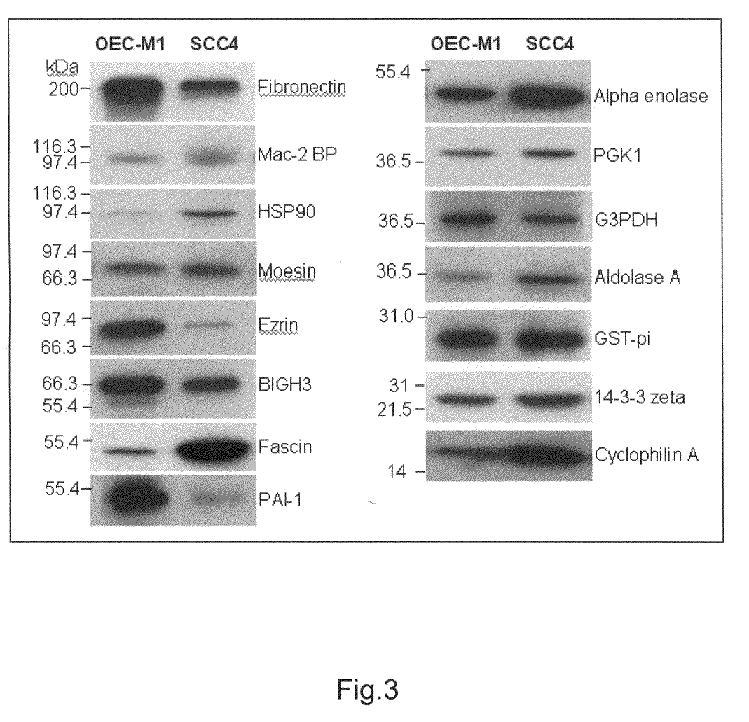

To verify the mass spectrometry-based protein identification, conditioned media from the two OSCC cell lines were subjected to Western blot analysis for 15 selected targets, using antibodies available commercially or produced in the laboratory. The selected targets primarily consisted of proteins that had been shown to be dysregulated in at least one cancer type and were detected by mass spectrometry in the secretomes of the two OSCC cell lines; these included fibronectin, Mac-2 binding protein (Mac-2 BP), HSP90, moesin, ezrin, TGF beta-induced protein BIGH3, fascin, plasminogen activator inhibitor 1 (PAI-1), alpha enolase, phosphoglycerate kinase 1 (PGK1), glyceraldehyde 3-phophate dehydrogenase (G3PDH), fructose-bisphosphate aldolase A, glutathione s-transferase P (GST-pi), 14-3-3 zeta and cyclophilin A. Proteins (30 μg) from the conditioned medium of the two OSCC cell lines were resolved in 8 or 12.5% SDS gels, transferred to a PVDF membrane, and then probed with specific antibodies against the indicated target proteins. As shown in FIG. 3, all of the target proteins were clearly detected in the conditioned media from the two oral cancer cell lines.

### Elevated Expression of Mac-2 BP in OSCC Specimens

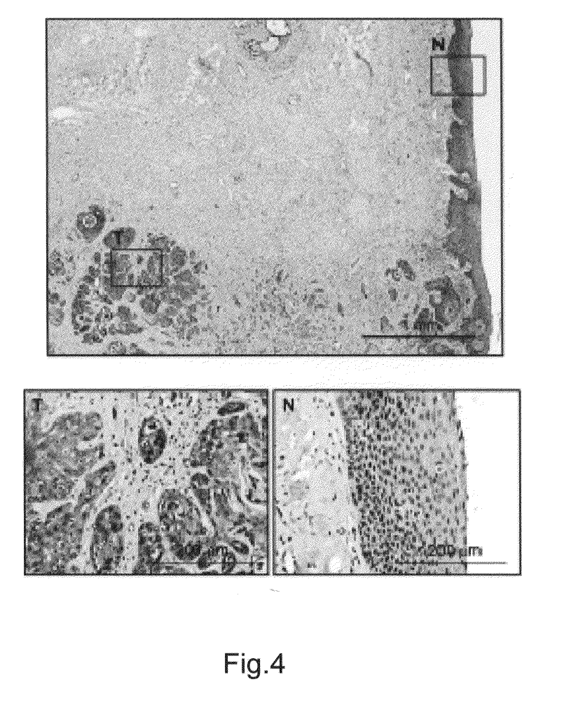

Among the 15 proteins confirmed by Western blot analysis, the inventor chose the cell adhesion-related protein Mac-2 BP for further evaluation in terms of its clinical relevance in OSCC. Dysregulation of Mac-2 BP has been reported in many cancer types, but has not be investigated in OSCC. The inventor herein examined the expression of Mac-2 BP in 146 OSCC patients, using immunohistochemistry to test tissue specimens containing both tumor and non-tumor cells. The clinicopathological characteristics of the 146 OSCC patients enrolled in this study are shown in Table 3. The inventor detected positive Mac-2 BP staining of tumor cells in 111 (76.3%) cases, whereas only 43 (29.5%) cases showed positive staining of adjacent non-tumor cells (Table 4). Among the 111 cases that harbored Mac-2 BP-positive tumor cells, 72 cases (64.9%) were negative for Mac-2 BP expression in their adjacent non-tumor cells (Table 3). Among the 35 cases harboring Mac-2 BP-negative tumor cells, most (˜90%, 31 out of 35) showed adjacent non-tumor cells that were also negative for Mac-2 BP expression (Table 3). One representative case of positive Mac-2 BP staining in tumor cells is shown in FIG. 4. In FIG. 4, Immunohistochemical staining of Mac-2 BP in OSCC specimens. OSCC specimens containing tumor (T) and adjacent non-tumor cells (N) were stained with a specific antibody against Mac-2 BP; one representative case is shown. The T and N areas indicated in upper panel (original magnification, ×40; scale bar, 1 mm) are enlarged and shown in lower panels (original magnification, ×200; scale bar, 200 μm). Clearly, the antibody significantly stained the cytoplasm of tumor cells, but showed little or no staining of adjacent non-tumor epithelial cells. These observations indicate that Mac-2 BP is overexpressed in OSCC tissues.

TABLE 3
Clinicopathological characteristics of the 146 OSCC patients
enrolled in this study.

| Characteristics | |
|---|---|
| Age (year, mean ± SD) | 50.7 ± 10.9 (rang 29-77) |
| SEX [n %] | Male 137 (93.84) |
| | Female 9 (6.16) |
| Site of primary tumor [n %] | Lip 3 (2.06) |
| | Oral cavity 128 (87.67) |
| | Oropharynx; Hypopharynx 15 |
| | (10.27) |
| Clinical stage [n %] | Stage I 13/143 (9.09) |
| | Stage II 37/143 (25.87) |
| | Stage III 19/143 (13.29) |
| | Stage IV 74/143 (51.75) |
| Regional lymph nodes [n %] | TNM-N0 76/121 (62.8) |
| | TNM-N1.N2 45/121 (37.2) |
| Cigarette smoker [n %] | 130 (98.66) |
| Alcohol drinker [n %] | 86 (59.31) |
| Betel quid chewer [n %] | 122 (84.14) |

TABLE 4
Expression of Mac-2 BP in 146 OSCC tissue specimens.

| | Case No. for | | Case No. for | | |
|---|---|---|---|---|---|
| | Mac-2 BP (+) | | Mac-2 BP (−) | | Total |
| | in tumor | | in tumor | | cases |
| | cells | | cells | | (%) |
| Case No. for Mac-2 | 39 | | 4 | | 43 (29.5) |
| BP (+) in adjacent non- | | | | | |
| tumor cells | | | | | |
| Case No. for Mac-2 BP (−) | 72 | | 31 | | 103 (70.5) |
| in adjacent non-tumor cells | | | | | |
| Total cases (%) | 111 | (76.3) | 35 | (23.7) | |

### Elevated Serum Levels of Mac-2 BP in OSCC Patients Versus Healthy Controls

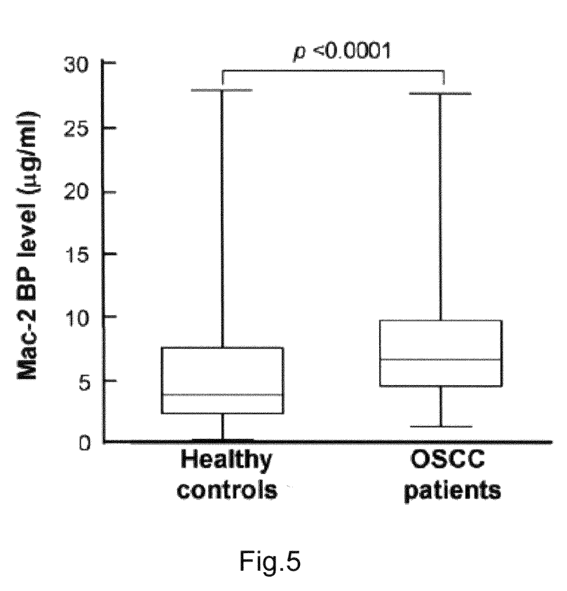

As mentioned, proteins upregulated in tumor tissues may or may not be detectable in accessible body fluids such as plasma and serum. However, proteins secreted by cancer cells could represent good serum/plasma biomarker candidates. The inventor previously developed a sensitive fluorimetric sandwich ELISA for Mac-2 BP that could be used to measure its level in blood samples (10). Here, the inventor used this method to examine whether the levels of Mac-2 BP were increased in the sera of OSCC patients versus healthy controls. The clinicopathological characteristics of the 88 OSCC patients and 106 healthy controls that provided serum samples in this study are shown in Table 5. In FIG. 5, Serum levels of Mac-2 BP in healthy controls and OSCC patients. The serum levels of Mac-2 BP in healthy controls (n=106) and OSCC patients (n=91) were measured by ELISA using 0.5 μl of serum. Data are presented as the upper and lower quartile and range (box), the median value (horizontal line), and the middle 90% distribution (dashed line). The inventor found that the serum levels of Mac-2 BP were significantly higher in OSCC patients (n=106) versus those in healthy controls (n=91) (mean±SD, 8.06±5.76 vs. 5.54±5.1 μg/ml; p<0.0001).

TABLE 5
Clinicopathological characteristics of the 88 OSCC patients
and 106 healthy controls that provided serum samples in this study.

| Characteristics | OSCC patients | | Healthy controls |
|---|---|---|---|
| Age (years, mean ± SD) | 48.9 ± 10.8 | (range | 56.0 ± 8.3 |
| | | 29-74) | (range 40-72) |
| Sex [n (%)] | | | |
| Male | 88 | (100) | 106 (100) |
| Female | 0 | (0) | 0 (0) |
| Site of primary tumor [n | | | |
| (%)] | | | |
| Lip | 0 | (0) | — |
| Oral cavity | 75 | (85.2) | — |
| Oropharynx; Hypopharynx | 13 | (14.8) | — |
| Clinical stage [n %] | | | |
| Stage I | 5/85 | (5.9) | — |
| Stage II | 21/85 | (24.7) | — |
| Stage III | 11/85 | (12.9) | — |
| Stage IV | 48/85 | (56.5) | — |
| Regional lymph nodes [n | | | |
| (%)] | | | |
| TNM-N0 | 59/86 | (68.6) | — |
| TNM-N1.N2 | 27/86 | (31.4) | — |
| Cigarette smoker [n (%)] | 82 | (93.2) | 59 (55.7) |
| Alcohol drinker [n (%)] | 58 | (65.9) | 35 (33.0) |
| Betel quid chewer [n (%)] | 78 | (88.6) | 43 (40.6) |

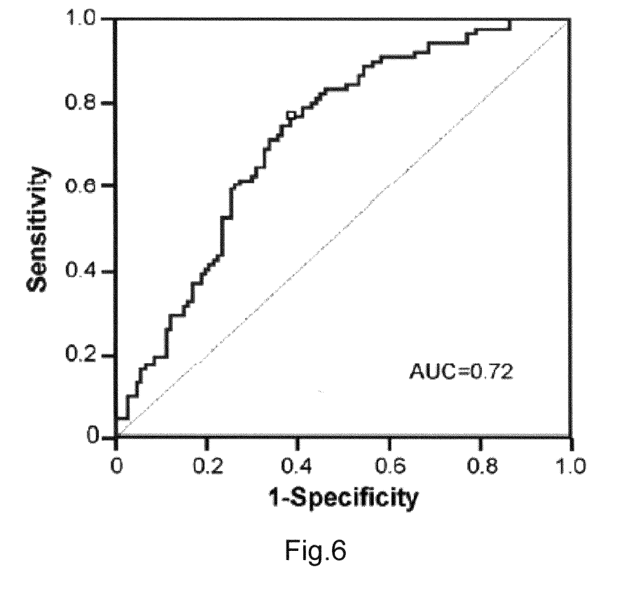

Based on this finding, the inventor then examined the diagnostic efficacy of Mac-2 BP by receiver operating characteristic (ROC) curve analysis. The area under the ROC curve (AUC) was determined to be 0.72 (95% CI, 0.64-0.78) for Mac-2 BP (FIG. 6). When applied a cut-off value of 4.45 μg/ml for Mac-2 BP to discriminate OSCC patients from healthy controls, the sensitivity and specificity values were 76.9% and 60.4%, respectively. These results indicate that Mac-2 BP is a potential serum biomarker for OSCC, and suggest the possible use of serum Mac-2 BP levels as a supplementary tool to aid oral cancer detection or monitoring.

### Materials and Methods

### Cell Culture

OEC-M1 is an oral epidermal carcinoma cell line derived from the gingiva of a Chinese patient (25), whereas SCC4 is a tongue squamous cell carcinoma cell line derived from a 55-year-old male (ATCC No. CRL-1624). The two cell lines were grown in RPMI medium containing 10% fetal bovine serum (FBS), 25 mM HEPES and antibiotics at 37° C. in 5% CO₂.

Harvest of Conditioned Media from Cancer Cell Lines

OEC-M1 and SCC4 cells were grown to confluence in 15-cm tissue culture dishes, and then washed twice with serum-free medium and incubated in serum-free medium for 24 hr. The conditioned media were harvested, centrifuged for elimination of intact cells, and concentrated by centrifugation in Amicon Ultra-15 tubes (molecular weight cutoff 5,000 Da; Millipore, Billerica, Mass.) three times at 4,000×g for 35 minutes each time. The concentrated samples were dried by Speed-Vac and resuspended in 100 μl deionized water for further use. The cells remaining on the dishes were washed twice with phosphate-buffered saline (PBS), and cell extracts were prepared as previously described (10, 26, 27). The protein concentrations of samples were determined using the BCA protein assay reagent from Pierce (Rockford, Ill.).

### Mass Spectrometric Analysis of Gel-Fractionated Proteins

Proteins were resolved on 9-15% gradient SDS gels and subjected to silver staining, and images were captured using a Personal Densitometer SI (Molecular Dynamics, Amersham Biosciences, Piscataway, N.J.). Protein bands were excised, destained and subjected to trypsin digestion as previously described (10, 26). Briefly, gel pieces were destained in 1% potassium ferricyanide and 1.6% sodium thiosulfate (Sigma, St. Louis, Mo.), dehydrated in acetonitrile and dried in a SpeedVac. The proteins were then reduced with 25 mM NH₄HCO₃ containing 10 mM dithiothreitol (Biosynth AG, Staad, Switzerland) at 60° C. for 30 min, and alkylated with 55 mM iodoacetamide (Amersham Biosciences) at room temperature for 30 min. After reduction and alkylation, the proteins were digested with sequencing-grade modified porcine trypsin (20 μg/ml) (Promega, Madison, Wis.) overnight at 37° C. The resulting peptides were extracted with acetonitrile containing 0.1% trifluoroacetic acid (v/v), and loaded onto an MTP AnchorChip™ 600/384 TF (Bruker-Daltonik GmbH, Bremen, Germany). MALDI-TOF mass spectrometry was performed on an Ultraflex™ MALDI-TOF mass spectrometer (Bruker Daltonik GmbH, Bremen, Germany). Peptide mass fingerprints were acquired in reflectron mode (26.7 kV accelerating voltage) with 300 laser shots per spectrum.

### Database Search and Protein Identification

Masses were automatically annotated using the Bruker Daltonics FlexAnalysis 2.2 software package (peak detection algorithm=SNAP; signal-to-noise threshold=2; maximal number of peaks=200; peak width=0.75 m/z; and quality factor threshold=50) and calibrated internally to a mass accuracy within 50 ppm, using a peptide mixture of bovine serum albumin (BSA) (m/z 927.49), human angiotensin II (m/z 1046.54), and ACTH-(18-39) (m/z 2465.198). Annotated and calibrated peaks were searched against the National Center for Biotechnology's non-redundant (NCBInr) database (released April 2005; 2,506,589 sequences and 850,049,330 residues) using the BioTools 2.2 software (Bruker Daltonics) and the Mascot search engine (version 2.1, Matrix Science, London, UK). Mascot searches were restricted to the human taxonomy (134,728 sequences), and with ‘trypsin digestion allowing a carbamidomethyl cysteine’ given as a fixed modification, and ‘oxidized methionine’ given as a potential variable modification. One trypsin-missed cleavage was allowed, and the peptide mass tolerance was set to 50 ppm. The known peptide masses of keratins were excluded. Positive identification was accepted when the data satisfied the following criteria: (i) targets were obtained with statistically significant search scores (greater than 95% confidence interval, equivalent to Mascot expected value <0.05); and (ii) the peptide ions of the identified proteins accounted for the majority of the ions present in the mass spectra. If the available peptides matched multiple members of a protein family in a Mascot search, the member with the highest ranked hit was selected. MS spectra with multiple matches were manually inspected to ensure the correct peptide-mass-fingerprint (PMF) assignment. Identified proteins were further analyzed using various software programs, including SignalP for predicting the presence of secretory signal peptide sequences (SignalP probability≧0.90)(28, 29), and SecretomeP for examining non-signal peptide-triggered protein secretion (SignalP probability<0.90 and SecretomeP score≧0.50)(30).

### Production of Antibodies

Anti-Mac-2 BP (120) and anti-heat shock protein 90 antibodies were produced in rabbits as previously described (10, 31). The antibody against BIGH3 was produced in rabbits using the peptide TQLYTDRTEKLRPEMEG(C), which corresponds to residues 118 to 134 of human BIGH3 (GenBank accession No. NM_{—}000358). This peptide was synthesized by Kelowna International Scientific Inc. (Taipei, Taiwan). A cysteine residue was added to the C-terminus to facilitate coupling of the peptide to BSA (Sigma). The antibodies were produced and affinity purified according to previously described procedures (10).

### Western Blot Analysis

The prepared samples (20 μg protein) were separated by SDS-PAGE, transferred to polyvinylidene difluoride (PVDF) membranes (Millipore), and then probed with various antibodies as previously described (10, 26). The utilized antibodies included anti-fibronectin (Santa Cruz Biotechnology, Santa Cruz, Calif.), anti-Mac-2 BP (120), anti-fascin (Santa Cruz Biotechnology), anti-heat shock protein 90, anti-moesin (Santa Cruz Biotechnology), anti-Ezrin (Abcam, Cambridge, Mass.), anti-BIGH3, anti-PAI-1 (Santa Cruz Biotechnology), anti-alpha enolase (Santa Cruz Biotechnology), anti-PGK1 (Santa Cruz Biotechnology), anti-G3PDH (Santa Cruz Biotechnology), anti-aldolase A (Santa Cruz Biotechnology), anti-GST-pi (Chemicon, Billerica, Mass.), anti-14-3-3 zeta (Upstate, Charlottesville, Va.), anti-cyclophilin A (Abcam) and anti-β-tubulin (MDbio, Taipei, Taiwan). Proteins of interest were detected with alkaline phosphatase-conjugated goat anti-rabbit IgG antibodies (Santa Cruz Biotechnology) and visualized with the CDP-Star™ chemiluminescent substrate (Boehringer Mannheim, Mannheim, Germany), according to the manufacturer's protocol.

### Patient Population and Clinical Specimens

Tumor specimens were obtained from 146 OSCC patients diagnosed at the Chang Gung Memorial Hospital (Tao-Yuan, Taiwan, Republic of China) in 1999-2000. The demographic data for these patients are shown in Supplementary Table S1. Serum samples were collected from 106 healthy controls [106 men ranging from 41 to 72 years of age (mean age 56.2±8.3)] and 91 OSCC patients [88 men and 3 women ranging from 29 to 74 years of age (mean age 49.1±10.8)] at the Chang Gung Memorial Hospital in 1999-2000. The enrolled cases included 5 stage-T1, 22 stage-T2, 11 stage-T3, 50 stage-T4 and 3 unknown stage patients. The study was approved by the Medical Ethics and Human Clinical Trial Committee at Chang Gung Memorial Hospital.

### Immunohistochemistry

Tissue specimens were fixed with 10% formaldehyde, embedded in paraffin, and cut into 4-μm-thick sections. Staining for Mac-2 BP was carried out using the Envision-kit (DAKO Corp., Carpinteria, Calif.). The sections were deparaffinized with xylene, dehydrated with ethanol and then retrieved by boiling in 10 mM citrate buffer (pH 6.0) for 20 min. Endogenous peroxidase activities were inactivated with the Dual Endogenous Enzyme Block (DAKO Corp.) for 15 min at room temperature, and then the sections were blocked using the Antibody Diluent with Background Reducing Components (DAKO Corp.) for 30 min. The sections were incubated with rabbit polyclonal antibodies to Mac-2 BP (120) (50 μg/ml) overnight at 4° C., and then washed, and exposed to a peroxidase-conjugated secondary anti-rabbit antibody (DAKO Corp.) for 30 min at room temperature, followed by treatment with substrate-chromogen solution (DAKO Corp.) and a further incubation for 5-10 min at room temperature. Finally, the sections were counterstained with hematoxylin (DAKO Corp.), dehydrated and mounted. Immunohistochemistry (IHC) staining intensity and percentage were evaluated by a pathologist (Li-Yu Lee). The staining intensity was scored as 0 (no stain) or I (weak to strong), and the staining percentage was scored as 0 (0˜49%) or 1 (≧50%) (17). The two scores were multiplied by each other to get the final score. Positive staining was defined as a final score=1.

### Fluorimetric Sandwich ELISA of Mac-2 BP

ELISA measurement of Mac-2 BP was performed as previously described (10). Briefly, white polystyrene microtiter plates (Corning, N.Y., USA) were coated with rabbit anti-Mac-2 BP (120) (10 μg/ml in PBS, 50 μl/well) antibodies overnight. The plates were then washed with TTBS and blocked with 200 μl of ovalbumin (Sigma) (1 mg/ml in TTBS). Recombinant MAC-2 BP (MedSystems Diagnostics GmbH, Vienna, Austria) was used as a standard. The serum samples comprised 0.5 μl of serum diluted to 50 μl in PBS containing 1% BSA were added and incubated at 37° C. for 1 h; the plates were then washed with TTBS. Subsequently, mouse anti-Mac-2 BP (BMS146, MedSystems Diagnostics GmbH, Vienna, Austria) (10 μg/ml in PBS, 50 μl/well) antibodies were applied and incubated for 1 h. After washing, 50 μl of alkaline phosphatase-conjugated goat anti-mouse IgG (Santa Cruz Biotechnology) (diluted 2000-fold in TTBS) was added and incubated for 1 h. Substrate 4-Methylumbelliferyl phosphate (100 μM; 100 μl/well, Molecular Probes, Eugene, Oreg., USA) was added, and then fluorescence was measured with a time-resolved fluorometer (the Plate Chameleon, Hidex, Turku, Finland) (λ_{excitation} 355 nm, λᵣₑₘᵢₛₛᵢₒₙ: 460 nm).

### Statistical Analysis.

The SAS® software package (version 8.2, SAS Institute, Cary, N.C.) was used to manage patient data and for statistical analysis. Mean between-group values were compared using the Chi-square or Fisher's exact tests. Wilcoxon Scores were used for ELISA group analysis. All statistical tests were two-sided, and p values less than 5% were considered significant. The receiver operating characteristic (ROC) curve was constructed by plotting sensitivity versus (1-specificity), considering each observed value as a possible cutoff value. The area under the ROC curve (AUC) was calculated as a single measure for the discriminative efficacy of each marker (32,33).

Those described above are only to exemplify the present invention but not to limit the scope of the present invention. Any modification or variation according to the spirit of the present invention is to be also included within the scope of the present invention, which is based on the claims stated below.

### REFERENCE

(1) Iacobelli, S.; Arno, E.; D'Orazio, A.; Coletti, G. Detection of antigens recognized by a novel monoclonal antibody in tissue and serum from patients with breast cancer. Cancer Res. 1986, 46, 3005-3010.
(2) Iacobelli, S.; Sismondi, P.; Giai, M.; D'Egidio, M.; Tinari, N.; Amatetti, C.; Di, S. P.; Natoli, C. Prognostic value of a novel circulating serum 90K antigen in breast cancer. Br. J. Cancer. 1994, 69, 172-176.
(3) Koths, K.; Taylor, E.; Halenbeck, R.; Casipit, C.; Wang, A. Cloning and characterization of a human Mac-2-binding protein, a new member of the superfamily defined by the macrophage scavenger receptor cysteine-rich domain. J. Biol. Chem. 1993, 268, 14245-14249.
(4) Ullrich, A.; Sures, 1.; D'Egidio, M.; Jallal, B.; Powell, T. J.; Herbst, R.; Dreps, A.; Azam, M.; Rubinstein, M.; Natoli, C.; The secreted tumor-associated antigen 90K is a potent immune stimulator J. Biol. Chem. 1994, 269, 18401-18407.
(5) Sasaki, T.; Brakebusch, C.; Engel, J.; Timpl, R. Mac-2 binding protein is a cell-adhesive protein of the extracellular matrix which self assembles into ring-like structures and binds beta1 integrins, collagens and fibronectin. EMBO J. 1998, 17, 1606-1613.
(6) Fornarini, B.; D'Ambrosio, C.; Natoli, C.; Tinari, N.; Silingardi, V.; Iacobelli, S. Adhesion to 90K (Mac-2 BP) as a mechanism for lymphoma drug resistance in vivo. Blood 2000, 96, 3282-3285.
(7) Zeimet, A. G.; Natoli, C.; Herold, M.; Fuchs, D.; Windbichler, G.; Daxenbichler, G.; Iacobelli, S.; Dapunt, O.; Marth, C. Circulating immunostimulatory protein 90K and soluble interleukin-2-receptor in human ovarian cancer. Int. J. Cancer. 1996, 68, 34-38.
(8) Marchetti, A.; Tinari, N.; Buttitta, F.; Chella, A.; Angeletti, C. A.; Sacco, R.; Mucilli, F.; Ullrich, A.; Iacobelli, S. Expression of 90K (Mac-2 BP) correlates with distant metastasis and predicts survival in stage I non-small cell lung cancer patients. Cancer Res. 2002, 62, 2535-2539.
(9) Ulmer, T. A.; Keeler, V.; Loh, L.; Chibbar, R.; Torlakovic, E.; Andre, S.; Gabius, H. J.; Laferte, S. Tumor-associated antigen 90K/Mac-2-binding protein: possible role in colon cancer. J. Cell Biochem. 2006, 98, 1351-1366.
(10) Wu, C. C.; Chien, K. Y.; Tsang, N. M.; Chang, K. P.; Ho, S. P.; Tsao, C. H.; Chang, Y. S.; Yu, J. S. Cancer cell-secreted proteomes as a basis for searching potential tumor markers—nasopharyngeal carcinoma as a model. Proteomics 2005, 5, 3173-3182.
(11) Artini, M.; Natoli, C.; Tinari, N.; Costanzo, A.; Marinelli, R.; Balsano, C.; Porcari, P.; Angelucci, D.; D'Egidio, M.; Levrero, M.; Iacobelli, S. Elevated serum levels of 90K/MAC-2 BP predict unresponsiveness to alpha-interferon therapy in chronic HCV hepatitis patients. J Hepatol. 1996, 25, 212-217.
(12) Gröschel B, Braner J J, Funk M, Linde R, Doerr H W, Cinatl J Jr, Iacobelli S. Elevated plasma levels of 90K (Mac-2 BP) immunostimulatory glycoprotein in HIV-1-infected children. J Clin Immunol. March 2000;20(2):117-22.
(13) Zeimet, A. G.; Natoli, C.; Herold, M.; Fuchs, D.; Windbichler, G.; Daxenbichler, G.; Iacobelli, S.; Dapunt, O.; Marth, C. Circulating immunostimulatory protein 90K and soluble interleukin-2-receptor in human ovarian cancer. Int. J. Cancer. 1996, 68, 34-38.
(14) Marchetti, A.; Tinari, N.; Buttitta, F.; Chella, A.; Angeletti, C. A.; Sacco, R.; Mucilli, F.; Ullrich, A.; Iacobelli, S. Expression of 90K (Mac-2 BP) correlates with distant metastasis and predicts survival in stage I non-small cell lung cancer patients. Cancer Res. 2002, 62, 2535-2539.
(15) Zhang, D. S.; Jiang, W. Q.; Li, S.; Zhang, X. S.; Mao, H.; Chen, X. Q.; Li, Y. H.; Zhan, J.; Wang, F. H. Predictive significance of serum 90K/Mac-2BP on chemotherapy response in non-Hodgkin's lymphoma. Ai Zheng. 2003, 22, 870-873.
(16) Iacobelli, S.; Sismondi, P.; Giai, M.; D'Egidio, M.; Tinari, N.; Amatetti, C.; Di, Stefano P.; Natoli, C. Prognostic value of a novel circulating serum 90K antigen in breast cancer. Br J Cancer. 1994, 69, 172-176.
(17) Marchetti, A.; Tinari, N.; Buttitta, F.; Chella, A.; Angeletti, C. A.; Sacco, R.; Mucilli, F.; Ullrich, A.; Iacobelli, S. Expression of 90K (Mac-2 BP) correlates with distant metastasis and predicts survival in stage I non-small cell lung cancer patients. Cancer Res. 2002, 62, 2535-2539.
(18) Nickel, W. The mystery of nonclassical protein secretion. A current view on cargo proteins and potential export routes. Eur. J. Biochem. 2003, 270, 2109-2119.
(19) Pisitkun, T.; Shen, R. F.; Knepper, M. A. Identification and proteomic profiling of exosomes in human urine. Proc. Natl. Acad. Sci. U.S.A. 2004, 101, 13368-13373.
(20) Mears, R.; Craven, R. A.; Hanrahan, S.; Totty, N.; Upton, C.; Young, S. L.; Patel, P.; Selby, P. J.; Banks, R. E. Proteomic analysis of melanoma-derived exosomes by two-dimensional polyacrylamide gel electrophoresis and mass spectrometry. Proteomics 2004, 4, 4019-4031.
(21) Chen, J.; He, Q. Y.; Yuen, A. P.; Chiu, J. F. Proteomics of buccal squamous cell carcinoma: the involvement of multiple pathways in tumorigenesis. Proteomics 2004, 4, 2465-2475. (22) Turhani, D.; Krapfenbauer, K.; Thurnher, D.; Langen, H.; Fountoulakis, M. Identification of differentially expressed, tumor-associated proteins in oral squamous cell carcinoma by proteomic analysis. Electrophoresis 2006, 27, 1417-1423.
(23) Lo, W. Y.; Tsai, M. H.; Tsai, Y.; Hua, C. H.; Tsai, F. J.; Huang, S. Y.; Tsai, C. H.; Lai, C. C. Identification of over-expressed proteins in oral squamous cell carcinoma (OSCC) patients by clinical proteomic analysis. Clinica Chimica Acta. 2007, 376, 101-107.
(24) Bakera, H.; Patel, V.; Molinolo, A. A.; Shillitoe, E. J.; Ensley, J. F.; Yoo, G. H.; Meneses-Garcia, A.; Myers, J. N.; El-Naggar, A. K.; Gutkind, J. S.; Hancock, W. S. Proteome-wide analysis of head and neck squamous cell carcinomas using laser-capture microdissection and tandem mass spectrometry. Oral Oncol. 2005, 41, 183-199.
(25) Meng, C. L.; Chao, C. F.; Tu, C. L.; Chang, L. C. Establishment, and characterization of a human oral epidermoid carcinoma cell line. Chin Dent J. 1984, 4, 103-105.
(26) Wu, C. C.; Chen, H. C.; Chen, S. J.; Liu, H. P.; Hsieh, Y. Y.; Yu, C. J.; Tang, R.; Hsieh, L. L.; Yu, J. S.; Chang, Y. S. (2008) Identification of collapsin response mediator protein-2 as a potential marker of colorectal carcinoma by comparative analysis of cancer cell secretomes. Proteomics 8, (2), 316-32.
(27) Wu, C. C.; Huang, Y. S.; Lee, L. Y.; Liang, Y.; Tang, R. P.; Chang, Y. S.; Hsieh, L. L.; Yu, J. S. (2008) Overexpression and elevated plasma level of tumor-associated antigen 90K/Mac-2 binding protein in colorectal carcinoma. Proteomics-Clinical Application 2, (12), 1586-95.
(28) Bendtsen, J. D.; Nielsen, H.; von, H. G.; Brunak, S. Improved prediction of signal peptides: SignalP 3.0. J. Mol. Biol. 2004, 340, 783-795.
(29) Nielsen, H.; Krogh, A. Prediction of signal peptides and signal anchors by a hidden Markov model. Proc. Int. Conf. Intell. Syst. Mol. Biol. 1998, 6, 122-130.
(30) Noh, D. Y.; Ahn, S. J.; Lee, R. A.; Kim, S. W.; Park, I. A.; Chae, H. Z. Overexpression of peroxiredoxin in human breast cancer. Anticancer Res. 2001, 21, 2085-2090.
(31) Huang, H. C.; Yu, J. S.; Tsay, C. C.; Lin, J. H.; Huang, S. Y.; Fang, W. T.; Liu, Y. C.; Tzang, B. S.; Lee, W. C. Purification and characterization of porcine testis 90-kDa heat shock protein (HSP90) as a substrate for various protein kinases. J. Protein Chem. 2002, 21, 111-121.
(32) Hanley, J. A.; McNeil, B. J. The meaning and use of the area under a receiver operating characteristic (ROC) curve. Radiology 1982, 143, 29-36.
(33) Zweig, M. H.; Campbell, G. Receiver-operating characteristic (ROC) plots: a fundamental evaluation tool in clinical medicine. Clin. Chem. 1993, 39, 561-577.

### Supporting Information (for Table 2)

(34) Kodama, J.; Hashimoto, I.; Seki, N. et al. Thrombospondin-1 and -2 messenger RNA expression in invasive cervical cancer: correlation with angiogenesis and prognosis. Clin Cancer Res. 2001, 7, 2826-2831.
(35) Kwak, C.; Jin, R. J.; Lee, C.; Park, M. S.; Lee, S. E. Thrombospondin-1, vascular endothelial growth factor expression and their relationship with p53 status in prostate cancer and benign prostatic hyperplasia. BJU Int. 2002, 89, 303-309.
(36) Grossfeld, G. D.; Carroll, P. R.; Lindeman, N. et al. Thrombospondin-1 expression in patients with pathologic stage T3 prostate cancer undergoing radical prostatectomy: association with p53 alterations, tumor angiogenesis, and tumor progression. Urology. 2002, 59, 97-102.
(37) Vallbo, C.; Wang, W.; Damber, J. E. The expression of thrombospondin-1 in benign prostatic hyperplasia and prostatic intraepithelial neoplasia is decreased in prostate cancer. BJU Int. 2004, 93, 1339-1343.
(38) Miyanaga, K.; Kato, Y,.; Nakamura, T. Expression and role of thrombospondin-1 in colorectal cancer. Anticancer Res. 2002, 22, 3941-3948.
(39) Albo, D.; Shinohara, T.; Tuszynski, G. P. Up-regulation of matrix metalloproteinase 9 by thrombospondin 1 in gastric cancer. J. Surg. Res. 2002, 108, 51-60.
(40) Yamaguchi, M.; Sugio, K.; Ondo, K.; Yano, T.; Sugimachi, K. Reduced expression of thrombospondin-1 correlates with a poor prognosis in patients with non-small cell lung cancer. Lung Cancer 2002, 36, 143-150.
(41) Urquidi, V.; Sloan, D.; Kawai, K. et al. Contrasting expression of thrombospondin-1 and osteopontin correlates with absence or presence of metastatic phenotype in an isogenic model of spontaneous human breast cancer metastasis. Clin. Cancer Res. 2002, 8, 61-74.
(42) Goddard, J. C.; Sutton, C. D.; Jones, J. L.; O'Byrne, K. J.; Kockelbergh, R. C. Reduced thrombospondin-1 at presentation predicts disease progression in superficial bladder cancer. Eur. Urol. 2002, 42, 464-468.
(43) Albo, D. and Tuszynski, G. P. Thrombospondin-1 up-regulates tumor cell invasion through the urokinase plasminogen activator receptor in head and neck cancer cells. J. Surg. Res. 2004, 20, 21-26.
(44) Gentiloni, N.; Caradonna, P.; Costamagna, G. Et al. Pancreatic juice 90K and serum CA 19-9 combined determination can discriminate between pancreatic cancer and chronic pancreatitis. Am. J. Gastroenterol. 1995, 90, 1069-1072.
(45) Künzli, B. M.; Berberat, P. O.; Zhu, Z. W. Et al. Influences of the lysosomal associated membrane proteins (Lamp-1, Lamp-2) and Mac-2 binding protein (Mac-2-BP) on the prognosis of pancreatic carcinoma. Cancer 2002, 94, 228-239.
(46) Marchetti, A.; Tinari, N.; Buttitta, F. Et al. Expression of 90K (Mac-2 BP) correlates with distant metastasis and predicts survival in stage I non-small cell lung cancer patients. Cancer Res. 2002, 62, 2535-2539.
(47) Iacovazzi, P. A.; Guerra, V.; Elba, S.; Sportelli, F.; Manghisi, O. G.; Correale, M. Are 90K/MAC-2BP serum levels correlated with poor prognosis in HCC patients? Preliminary results. Int. J. Biol. Markers 2003, 18,222-226.
(48) Wu, C. C.; Chien, K. Y.; Tsang, N. M.; Chang, K. P.; Hao, S. P.; Tsao, C. H.; Chang, Y. S.; Yu, J. S. Cancer cell-secreted proteomes as a basis for searching potential tumor markers: nasopharyngeal carcinoma as a model. Proteomics 2005, 5, 3173-3182.
(49) Bair, E. L.; Nagle, R. B.; Ulmer, T. A.; Laferté, S.; Bowden, G. T. 90K/Mac-2 binding protein is expressed in prostate cancer and induces promatrilysin expression. Prostate 2006, 66, 283-293.
(50) Ulmer, T. A.; Keeler, V.; Loh, L. Et al. Tumor-associated antigen 90K/Mac-2-binding protein: possible role in colon cancer. J. Cell. Biochem. 2006, 98, 1351-1366.
(51) Park, Y. P.; Choi, S. C.; Kim, J. H. et al. Up-regulation of Mac-2 binding protein by hTERT in gastric cancer. Int. J. Cancer 2007, 120, 813-820.
(52) Mukai T. Immunohistochemical study of fibronectin and laminin on gastric cancer. Nippon Gan Chiryo Gakkai Shi. 1990, 25, 2468-2476.
(53) Olt, G.; Berchuck, A.; Soisson, A. P.; Boyer, C. M.; Bast, R. C. Jr. Fibronectin is an immunosuppressive substance associated with epithelial ovarian cancer. Cancer 1992, 70, 2137-2142.
(54) Ioachim, E. E.; Athanassiadou, S. E.; Kamina, S.; Carassavoglou, K.; Agnantis, N. J. Matrix metalloproteinase expression in human breast cancer: an immunohistochemical study including correlation with cathepsin D, type IV collagen, laminin, fibronectin, EGFR, c-erbB-2 oncoprotein, p53, steroid receptors status and proliferative indices. Anticancer Res. 1998, 18, 1665-1670.
(55) Ioachim, E.; Charchanti, A.; Briasoulis, E. Et al. Immunohistochemical expression of extracellular matrix components tenascin, fibronectin, collagen type IV and laminin in breast cancer: their prognostic value and role in tumour invasion and progression. Eur. J. Cancer 2002, 38, 2362-2370.
(56) Warawdekar, U. M.; Zingde, S. M.; Iyer, K. S.; Jagannath, P.; Mehta, A. R.; Mehta, N. G. Elevated levels and fragmented nature of cellular fibronectin in the plasma of gastrointestinal and head and neck cancer patients. Clin. Chim. Acta 2006, 372, 83-93.
(57) Pietruszewska, W.; Kobos, J.; Bojanowska-Poźniak, K.; Durko, M.; Gryczyński, M. Immunohistochemical analysis of the fibronectin expression and its prognostic value in patients with laryngeal cancer. Otolaryngol. Pol. 2006, 60, 697-702.
(58) Nishizuka, S.; Chen, S. T.; Gwadry, F. G.; Alexander, J.; Major, S. M.; Scherf, U.; Reinhold, W. C.; Waltham, M.; Charboneau, L.; Young, L.; Bussey, K. J.; Kim, S.; Lababidi, S.; Lee, J. K.; Pittaluga, S.; Scudiero, D. A.; Sausville, E. A.; Munson, P. J.; Petricoin, E. F.; III, Liotta, L. A.; Hewitt, S. M.; Raffeld, M.; Weinstein, J. N. Diagnostic markers that distinguish colon and ovarian adenocarcinomas: identification by genomic, proteomic, and tissue array profiling. Cancer Res. 2003, 63, 5243-5250.
(59) Kobayashi, H.; Sagara, J.; Kurita, H.; Morifuji, M.; Ohishi, M.; Kurashina, K.; Taniguchi, S. Clinical significance of cellular distribution of moesin in patients with oral squamous cell carcinoma. Clin. Cancer Res. 2004, 10, 572-580.
(60) Cardillo, M. R.; Sale, P.; Di Silverio, F. Heat shock protein-90, IL-6 and IL-10 in bladder cancer. Anticancer Res. 2000, 20, 4579-4583.
(61) Cardillo, M. R.; Ippoliti, F. IL-6, IL-10 and HSP-90 expression in tissue microarrays from human prostate cancer assessed by computer-assisted image analysis. Anticancer Res. 2006, 26, 3409-3416.
(62) Pick, E.; Kluger, Y.; Giltnane, J. M.; Moeder, C.; Camp, R. L.; Rimm, D. L.; Kluger, H. M. High HSP90 expression is associated with decreased survival in breast cancer. Cancer Res. 2007, 67, 2932-2937.
(63) Buckhaults, P.; Rago, C.; St Croix, B.; Romans, K. E.; Saha, S.; Zhang, L.; Vogelstein, B.; Kinzler, K. W. Secreted and cell surface genes expressed in benign and malignant colorectal tumors. Cancer Res. 2001, 61, 6996-7001.
(64) Schneider, D.; Kleeff, J.; Berberat, P. O.; Zhu, Z.; Korc, M.; Friess, H.; Buchler, M. W. Induction and expression of betaig-h3 in pancreatic cancer cells. Biochim. Biophys. Acta 2001, 1588, 1-6.
(65) Hu, Y. C.; Lam, K. Y.; Law, S.; Wong, J.; Srivastava, G. Profiling of differentially expressed cancer-related genes in esophageal squamous cell carcinoma (ESCC) using human cancer cDNA arrays: overexpression of oncogene MET correlates with tumor differentiation in ESCC. Clin. Cancer Res. 2001, 7, 3519-3525.
(66) Takashima, M.; Kuramitsu, Y.; Yokoyama, Y. et al. Overexpression of alpha enolase in hepatitis C virus-related hepatocellular carcinoma: association with tumor progression as determined by proteomic analysis. Proteomics 2005, 5, 1686-92.
(67) Chang, G. C.; Liu, K. J.; Hsieh, C. L.; Hu, T. S.; Charoenfuprasert, S.; Liu, H. K.; Luh, K. T.; Hsu, L. H.; Wu, C. W.; Ting, C. C.; Chen, C. Y.; Chen, K. C.; Yang, T. Y.; Chou, T. Y.; Wang, W. H.; Whang-Peng, J.; Shih, N. Y. Identification of alpha-enolase as an autoantigen in lung cancer: its overexpression is associated with clinical outcomes. Clin. Cancer Res. 2006, 12, 5746-5754.
(68) Ito, S.; Honma, T.; Ishida, K.; Wada, N.; Sasaoka, S.; Hosoda, M.; Nohno, T. Differential expression of the human alpha-enolase gene in oral epithelium and squamous cell carcinoma. Cancer Sci. 2007, 98, 499-505.
(69) Hu, W.; McCrea, P. D.; Deavers, M.; Kavanagh, J. J.; Kudelka, A. P.; Verschraegen, C. F. Increased expression of fascin, motility associated protein, in cell cultures derived from ovarian cancer and in borderline and carcinomatous ovarian tumors. Clin. Exp. Metastasis 2000, 18, 83-88.
(70) Iacobuzio-Donahue, C. A.; Ashfaq, R.; Maitra, A. et al. Highly expressed genes in pancreatic ductal adenocarcinomas: a comprehensive characterization and comparison of the transcription profiles obtained from three major technologies. Cancer Res. 2003, 63, 8614-8622.
(71) Pelosi, G.; Pastorino, U.; Pasini. F. et al. Independent prognostic value of fascin immunoreactivity in stage I nonsmall cell lung cancer. Br. J. Cancer 2003, 88, 537-547.
(72) Peraud, A.; Mondal, S.; Hawkins, C.; Mastronardi, M.; Bailey, K.; Rutka, J. T. Expression of fascin, an actin-bundling protein, in astrocytomas of varying grades. Brain Tumor Pathol. 2003, 2053-2058.
(73) Yoder, B. J.; Tso, E.; Skacel, M.; Pettay, J.; Tarr, S.; Budd, T.; Tubbs, R. R.; Adams, J. C.; Hicks, D. G. The expression of fascin, an actin-bundling motility protein, correlates with hormone receptor-negative breast cancer and a more aggressive clinical course. Clin. Cancer Res. 2005, 11, 186-192.
(74) Hashimoto, Y.; Skacel, M.; Lavery, I. C.; Mukherjee, A. L.; Casey. G.; Adams, J. C. Prognostic significance of fascin expression in advanced colorectal cancer: an immunohistochemical study of colorectal adenomas and adenocarcinomas. BMC Cancer 2006, 6:241.
(75) Jin, J. S.; Yu, C. P.; Sun, G. H.; Lin, Y. F.; Chiang, H.; Chao, T. K.; Tsai, W. C.; Sheu, L. F. Increasing expression of fascin in renal cell carcinoma associated with clinicopathological parameters of aggressiveness. Histol. Histopathol. 2006, 21, 1287-1293.
(76) Zhang, H.; Xu, L.; Xiao, D.; Xie, J.; Zeng, H.; Cai, W.; Niu, Y.; Yang, Z.; Shen, Z.; Li, E. Fascin is a potential biomarker for early-stage oesophageal squamous cell carcinoma. J. Clin. Pathol. 2006 59, 958-964.
(77) Lee, T. K.; Poon, R. T.; Man, K.; Guan, X. Y.; Ma, S.; Liu, X. B.; Myers, J. N.; Yuen, A. P. Fascin over-expression is associated with aggressiveness of oral squamous cell carcinoma. Cancer Lett. 2007, 254, 308-315.
(78) Foekens, J. A.; Schmitt. M.; van Putten. W. L. et al. Plasminogen activator inhibitor-1 and prognosis in primary breast cancer. J. Clin. Oncol. 1994, 12, 1648-1658.
(79) Costantini, V.; Sidoni, A.; Deveglia, R.; Cazzato, O. A.; Bellezza, G.; Ferri, I.; Bucciarelli, E.; Nenci, G. G. Combined overexpression of urokinase, urokinase receptor, and plasminogen activator inhibitor-1 is associated with breast cancer progression: an immunohistochemical comparison of normal, benign, and malignant breast tissues. Cancer 1996, 77, 1079-1088.
(80) Pavey, S. J.; Marsh, N. A.; Ray, M. J.; Butler, D.; Dare, A. J.; Hawson, G. A. Changes in plasminogen activator inhibitor-1 levels in non-small cell lung cancer. Boll. Soc. Ital. Biol. Sper. 1996, 72, 331-340.
(81) Kawasaki, K.; Hayashi, Y.; Wang, Y. et al. Expression of urokinase-type plasminogen activator receptor and plasminogen activator inhibitor-1 in gastric cancer. J. Gastroenterol. Hepatol. 1998, 13, 936-944.
(82) Nielsen, H. J.; Pappot, H.; Christensen, I. J.; et al. Association between plasma concentrations of plasminogen activator inhibitor-1 and survival in patients with colorectal cancer. BMJ 1998, 316, 829-830.
(83) Pasini, F. S.; Brentani, M. M.; Kowalski, L. P.; Federico, M. H. Transforming growth factor beta1, urokinase-type plasminogen activator and plasminogen activator inhibitor-1 mRNA expression in head and neck squamous carcinoma and normal adjacent mucosa. Head Neck 2001, 23,725-732.
(84) Sakakibara, T.; Hibi, K.; Kodera, Y.; Ito, K.; Akiyama, S.; Nakao, A. Plasminogen activator inhibitor-1 as a potential marker for the malignancy of esophageal squamous cell carcinoma. Clin. Cancer Res. 2004, 10, 1375-1378.
(85) Wu, C. C.; Chien, K. Y.; Tsang, N. M; Chang, K. P.; Hao, S. P.; Tsao, C. H.; Chang, Y. S.; Yu, J. S. Cancer cell-secreted proteomes as a basis for searching potential tumor markers: nasopharyngeal carcinoma as a model. Proteomics 2005, 5, 3173-3182.
(86) Sakakibara, T.; Hibi, K.; Koike, M.; Fujiwara, M.; Kodera, Y.; Ito, K.; Nakao, A. Plasminogen activator inhibitor-1 as a potential marker for the malignancy of gastric cancer. Cancer Sci. 2006, 97, 395-399.
(87) Speleman, L.; Kerrebijn, J. D.; Look, M. P.; Meeuwis, C. A.; Foekens, J. A.; Berns, E. M. Prognostic value of plasminogen activator inhibitor-1 in head and neck squamous cell carcinoma. Head Neck 2007, 29, 341-350.
(88) Hirata, S.; Odajima, T.; Kohama, G.; Ishigaki, S.; Niitsu, Y. Significance of glutathione S-transferase-pi as a tumor marker in patients with oral cancer. Cancer 1992, 70, 2381-2387.
(89) Howie, A. F. Measurement of glutathione S-transferase pi by radioimmunoassay: elevated plasma levels in lung cancer patients. Br. J. Biomed. Sci. 1993, 50, 187-199.
(90) Hida, T.; Kuwabara, M.; Ariyoshi, Y. et al. Serum glutathione S-transferase-pi level as a tumor marker for non-small cell lung cancer. Potential predictive value in chemotherapeutic response. Cancer 1994, 7, 1377-8132.
(91) Monden, N.; Abe, S.; Sutoh, I.; Hishikawa, Y.; Kinugasa, S.; Nagasue, N. Prognostic significance of the expressions of metallothionein, glutathione-S-transferase-pi, and P-glycoprotein in curatively resected gastric cancer. Oncology 1997, 5, 391-319
(92) Berendsen, C. L.; Mulder, T. P.; Peters, W. H. Plasma glutathione S-transferase pi 1-1 AND alpha 1-1 levels in patients with bladder cancer. J. Urol. 2000, 164, 2126-2128.
(93) Jayasurya, A.; Yap, W. M.; Tan, N. G.; Tan, B. K.; Bay, B. H. Glutathione S-transferase pi expression in nasopharyngeal cancer. Arch. Otolaryngol. Head Neck Surg. 2002, 128, 1396-1399.
(94) Huang, J.; Tan, P. H.; Thiyagarajan, J.; Bay, B. H. Prognostic significance of glutathione S-transferase-pi in invasive breast cancer. Mod. Pathol. 2003, 16, 558-565.
(95) Li, M.; Ittmann, M. M.; Rowley, D. R.; Knowlton, A. A.; Vaid, A. K.; Epner, D. E. Glutathione S-transferase pi is upregulated in the stromal compartment of hormone independent prostate cancer. Prostate 2003, 56, 98-105.
(96) Yanagawa, T.; Iwasa, S.; Ishii, T. et al. Peroxiredoxin I expression in oral cancer: a potential new tumor marker. Cancer Lett. 2000, 156, 27-35.
(97) Noh, D. Y.; Ahn, S. J.; Lee, R. A.; Kim, S. W.; Park, I. A.; Chae, H. Z. Overexpression of peroxiredoxin in human breast cancer. Anticancer Res. 2001, 21, 2085-2090.
(98) Kim, H. J.; Chae, H. Z; Kim, Y. J.; Kim, Y. H.; Hwangs, T. S.; Park, E. M.; Park, Y. M. Preferential elevation of Prx I and Trx expression in lung cancer cells following hypoxia and in human lung cancer tissues. Cell Biol. Toxicol 2003, 19, 285-298.
(99) Park, J. H.; Kim, Y. S.; Lee, H, L, et al. Expression of peroxiredoxin and thioredoxin in human lung cancer and paired normal lung. Respirology 2006, 11, 269-275.
(100) Chen, G.; Gharib, T. G.; Wang, H.; Huang, C. C.; Kuick, R.; Thomas, D. G.; Shedden, K. A.; Misek, D. E.; Taylor, J. M.; Giordano, T. J.; Kardia, S. L.; Iannettoni, M. D.; Yee, J.; Hogg, P. J.; Orringer, M. B.; Hanash, S. M.; Beer, D. G. Protein profiles associated with survival in lung adenocarcinoma. Proc. Natl. Acad. Sci. USA 2003, 98, 13790-13795.
(101) Liu, D. W.; Chen, S. T.; Liu, H. P. Choice of endogenous control for gene expression in nonsmall cell lung cancer. Eur. Respir. J. 2005, 26, 1002-1008.
(102) Hwang, T. L.; Liang, Y.; Chien, K. Y.; Yu, J. S. Overexpression and elevated serum levels of phosphoglycerate kinase 1 in pancreatic ductal adenocarcinoma. Proteomics 2006, 6, 2259-2272.
(103) Wang, J.; Wang, J.; Dai, J. et al. A lycolytic mechanism regulating an angiogenic switch in prostate cancer. Cancer Res. 2007, 67, 149-159.
(104) Li, C.; Xiao, Z.; Chen, Z.; Zhang, X.; Li, J.; Wu, X.; Li, X.; Yi, H.; Li, M.; Zhu, G.; Liang, S. Proteome analysis of human lung squamous carcinoma. Proteomics 2006, 6,547-558.
(105) Qi, W.; Liu, X.; Qiao, D.; Martinez, J. D. Isoform-specific expression of 14-3-3 proteins in human lung cancer tissues. Int. J. Cancer 2005, 113, 359-363.
(106) Fan, T.; Li, R.; Todd, N. W. et al. Up-regulation of 14-3-3zeta in lung cancer and its implication as prognostic and therapeutic target. Cancer Res. 2007, 67, 7901-7906.
(107) Matta, A.; Bahadur, S.; Duggal, R.; Gupta, S. D.; Ralhan, R. Over-expression of 14-3-3zeta is an early event in oral cancer. BMC Cancer 2007, 7, 169.
(108) Jang, J. S.; Cho, H. Y.; Lee, Y. J.; Ha, W. S.; Kim, H. W. The differential proteome profile of stomach cancer: identification of the biomarker candidates. Oncol. Res. 2004, 14, 491-49.

What is claimed is:

1. A biomarker for oral cancer diagnosis, which is Mca-2 binding protein (Mac-2BP) existing in body fluid of a testee.

2. The biomarker for oral cancer diagnosis according to claim 1, wherein said body fluid is plasma, serum or urine.

3. The biomarker for oral cancer diagnosis according to claim 1, wherein said oral cancer is oral squamous cell cancer.

4. A method for inspecting oral cancer, comprising detecting Mac-2BP expression level of a body fluid of a testee suspected to have oral cancer.

5. The method for inspecting oral cancer according to claim 4, wherein Mac-2BP expression level of a body fluid of a testee suspected to have oral cancer is compared with Mac-2BP expression level of a body fluid of health testees.

6. The method for inspecting oral cancer according to claim 4, wherein said Mac-2BP expression level is used as an indicator of existence of oral cancer or differentiation of oral cancer.

7. The method for inspecting oral cancer according to claim 4, wherein said body fluid is plasma, serum or urine.

8. The method for inspecting oral cancer according to claim 4, wherein concentration of Mac-2BP is detected with an immunoassay method.

9. The method for inspecting oral cancer according to claim 8, wherein said immunoassay method is an ELISA (Enzyme-Linked ImmunoSorbent Assay) method.

10. The method for inspecting oral cancer according to claim 7, wherein said oral cancer is oral squamous cell cancer.

11. An oral cancer inspection method using an oral cancer biomarker, comprising steps:
cultivating cell lines of oral cancer in a serum-free environment, and respectively collecting proteins secreted by said cell lines;
using 9-15% gradient electrophoresis to separate said proteins and staining said proteins with silver ion;
cutting off electrophoresis gel containing said protein having been stained, and using trypsin to hydrolyze said proteins in gel;
using MALDI-TOF (Matrix-Assisted Laser Desorption/Ionization-Time Of Flight) to analyze said proteins, which have been hydrolyzed, to identify identities of said proteins respectively secreted by said cell lines; and
performing analysis to find out said proteins that have been identified in said cell lines simultaneously, and using said proteins that have been identified in said cell lines simultaneously as biomarkers.

12. The oral cancer inspection method using an oral cancer biomarker according to claim 11, wherein said oral cancer biomarker is Mca-2 binding protein (Mac-2BP).
